(19) United States
(12) Patent Application Publication
(10) Pub. No.: US 2007/0043404 A1
(43) Pub. Date: Feb. 22, 2007

(54) Device for electrically stimulating parts of the nervous system

(75) Inventor: Michael Deimling, Mohrendorf (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/504,507

(22) Filed: Aug. 15, 2006

(30) Foreign Application Priority Data
Aug. 18, 2005 (DE) 10 2005 039 183.4

(51) Int. Cl. A61N 1/00

(52) U.S. Cl. 607/61

(57) ABSTRACT

A device is provided for electrically stimulating parts of the nervous system for the treatment of medical conditions. The device includes a generator, an intracorporeal electrode suitable for implantation in a human body and an MRT-compatible coupling. The MRT-compatible coupling couples the generator and the electrode in a manner that enables energy transfer from the generator to the electrode and which retains the generator and electrode in electrical isolation. The generator provides energy, such as in the form of light, to the electrode via the MRT-compatible coupling to generate and control the voltage in the electrode for stimulating parts of the nervous system. The generator can control the light intensity in order to adjust the strength of the electrode voltage.

### CROSS REFERENCE TO RELATED APPLICATIONS

This application claims priority of German application No. 102005039183.4 filed Aug. 18, 2005, which is incorporated by reference herein in its entirety.

### FIELD OF INVENTION

The invention relates to a device for electrically stimulating parts of the nervous system using a generator and at least one intracorporeal electrode.

### BACKGROUND OF INVENTION

Devices of this type are used with different neurological diseases. Within the scope of Morbus Parkinson therapy, electrodes are steriotactically implanted into the brain during the deep brain stimulation by means of a neurosurgical operation. A targeted electrical excitation of a brain area in the region of the basal ganglia located around the electrode tip allows typical Parkinson's disease symptoms such as tremor, rigor or akinesia to be significantly reduced. Implantable neurological stimulators are also used with other neurological diseases. Depending on the underlying disease and on the desired therapy, different regions of the nervous system, such as the brain, parts of the spinal cord, cranial nerve or also peripheral nerves are excited here by means of neurological stimulators. By way of example, certain forms of epilepsy can be positively influenced by a vagus nerve stimulator. With different pain syndromes, peripheral nerves, areas near to the spinal cord or intracranial regions are electrically excited by means of implantable neurological stimulators in order to improve the pain symptoms.

A device for electrically stimulating parts of the nervous system usually comprises intracorporeal electrodes at the site of the excitation and a generator, which is likewise mostly implanted intracorporeally, below the collar bone or in the abdomen for instance. In this way, the electrodes are connected to the generator via a current-conducting cable.

With patients with an implanted neurological stimulator, imaging methods, in particular magnetic resonance tomography examinations (MRT examinations), must often be carried out as a result of the neurological underlying disease. An increased risk of complication exists here by the use of the stimulator. With an MRT device, three different electromagnetic fields are usually radiated to produce the image; a static magnetic field, a gradient magnetic field and a high-frequency magnetic field. The static magnetic field usually exhibits a magnetic field strength of 0.2 to 3 tesla. The frequencies of the high-frequency magnetic field are attuned to the magnetic field strengths of the static magnetic field. Frequencies of the high-frequency magnetic field thus result, with which the current-conducting cables act as antennae for the high-frequency magnetic field and can heat up to more than 25° C. This represents a potential source of danger for patients. This danger even exists when the device is in a deactivated state during the MRT examination. In the FDA Public Health Notification: MRI-Caused Injuries in Patients with Implanted Neurological Stimulators, May 2005, an account is given of incidents in which patients with an implantable neurological stimulator have suffered serious, and sometimes permanent damage to the point of a coma, after an MRT examination.

US 2005/0070972 A1 describes a device for neurostimulation, in which an electrically conductive cable is affixed between the neurostimulator and the electrode,—the cable having an electrically coupled shunt connection in order to discharge the high-frequency energy radiated during an MRT examination from the part of the cable feeding towards the electrode. The shunt connection is preferably provided here with a high-frequency filter.

DE 103 55 652 A1 describes a device for desynchronizing neuronal, diseased synchronous brain activity, in which the activities are stimulated with at least two electrodes in at least two subareas of a brain area or at least two brain areas functionally belonging together, whereby with a diseased person, a desynchronization is adjusted in the affected neuron population and the symptoms are suppressed. The impulses are released via electrodes by means of a stimulator unit. The stimulator unit itself is controlled by a control unit, with the control signals being optically injected into the stimulator unit so that an interspersion of interference signals from the control unit into the electrodes is prevented.

### SUMMARY OF THE INVENTION

The object of the present invention is to embody a device for electrically stimulating parts of the nervous system in a simple manner such that the risk of patients suffering damage during an MRT examination is reduced.

This object is achieved in accordance with the invention by a device according to the independent claims. Advantageous embodiments of the invention are the subject matter of further dependent claims in each instance.

The device for electrically stimulating parts of the nervous system according to an independent claims comprises a generator and at least one intracoporeal electrode, with the generator and the at least one electrode being electrically isolated and being coupled via MRT-compatible coupling-enabling energy transport in accordance with the invention. MRT-compatible coupling is understood here to mean a coupling between the generator and the electrode, which conducts energy from the generator to the electrode, by means of which components however no risk exists for the patient as a result of excessive heating during an MRT examination. A device constructed in this way no longer requires a current-conducting cable to connect the generator to the electrode, which presents a common cause of complications during an MRT examination.

The electrode advantageously features at least one photovoltaic element and is controlled by the generator by means of light, which is conducted via a light-conducting cable from the generator to the electrode. This simple arrangement ensures the electrical isolation of the electrode and the generator with an MRT-compatible coupling. Here, the light-conducting cable can be a single fiber optic cable or also feature several fiber optic cables.

In a particularly simple embodiment, the photovoltaic element is designed as a plane surface which can be radiated with the light leaving from the light-conducting cable. In an especially space-saving arrangement, the photovoltaic element is embodied as a surface coaxially curved around the cable conducting the light. The electrodes, by means of which the tissue is excited, are affixed here to the photovoltaic element.

Advantageously the strength of the electrode voltage can be controlled via the light intensity generated by the generator. The electrode can hereby be generated by the generator in a simple manner and its functionality can be adjusted to the requirements.

In a simple and cost-effective embodiment, the light can be generated by a light-emitting diode. The wavelength of the light is attuned here to the photovoltaic element in order to achieve the largest possible degree of efficiency. Advantageously, blue light is used as a result of the high energy content, with the photovoltaic element then being attuned to the frequency range of the blue light.

In one embodiment of the device, the light can be generated as a continuous light signal by means of the generator. A uniform electrode voltage can be generated on the electrode with the aid of an embodiment of this type.

In another embodiment of the device, the light can be generated as an impulsed light signal by means of the generator. Voltage impulses on the electrode tips can be hereby emitted into the tissue.

The invention as well as further advantageous embodiments according to the features of the claims are described in more detail below in the drawing with reference to schematically represented exemplary embodiments, without however being restricted thereto, in which;

### BRIEF DESCRIPTION OF THE DRAWINGS

FIG. 1 shows a schematic representation of a human body, in which a device for electrically stimulating parts of the brain is implanted,

FIG. 2 shows a first embodiment of the electrode with a plane photovoltaic element and

FIG. 3 shows a further embodiment of the electrode with a photovoltaic element arranged coaxially around a light-conducting cable.

### DETAILED DESCRIPTION OF INVENTION

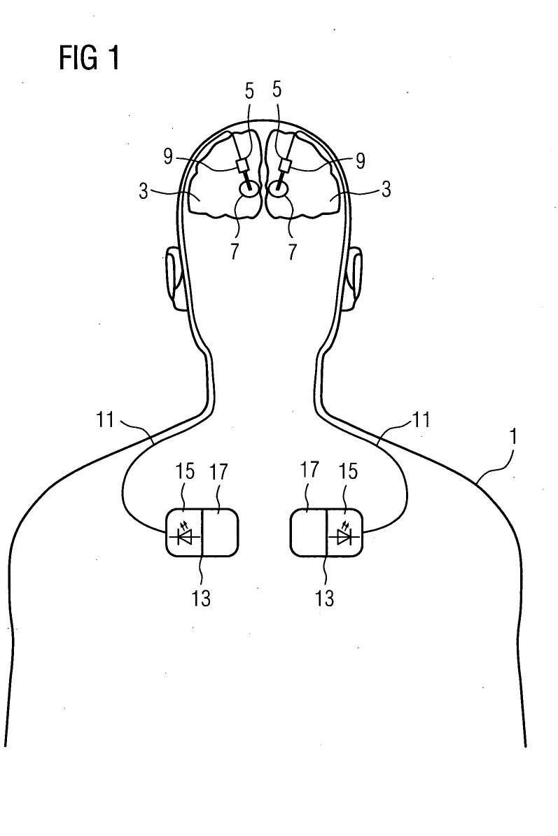

FIG. 1 shows a schematic display of the human body 1. Electrodes 5 are implanted into the brain 3, in order to purposefully electrically excite regions 7 of the brain 3. The electrode voltage required here supplies a photovoltaic element 9, which is operated with the aid of light impulses which are conducted over a light-conducting cable 11 from a generator 13 to the photovoltaic element 9. In the example shown here, the generator 13 is similarly implanted into the body 1 of the patient. The light-conducting cable is guided completely below the skin surface. The generator 13 can however also be found extracorporeally. In this case, the light-conducting cable 11 passes through the skin at a specific point.

Electrical energy is converted in the generator 13 into light energy, with the aid of a light diode 15 for instance, the light of which is guided into the light-conducting cable 11. In this way, the wavelength of the emitted light is attuned to the photovoltaic element 9 so as to achieve the greatest possible degree of efficiency. The intensity of the light of the light-emitting diode 15 and the electrode voltage are controlled by a control unit 17 which is located in the generator 13.

The arrangement shown here is suitable for implementing a deep brain stimulation in a patient. A therapy of this type is used for instance within the scope of a Morbus Parkinson therapy or a chronic pain syndrome therapy. The electrodes 5 can however also be placed in other regions of the nervous system. By way of example, the electrodes 5 can be placed along a cranial nerve, particularly here along the vagus nerve. An arrangement of this type is used in the therapy of certain forms of epilepsy.

With different pain symptoms, the electrodes 5 can be placed in the region of peripheral nerves or in the region of the spinal cord, epidurally, intrathecally or completely in the spinal cord itself so as to achieve an improvement in the pain symptoms.

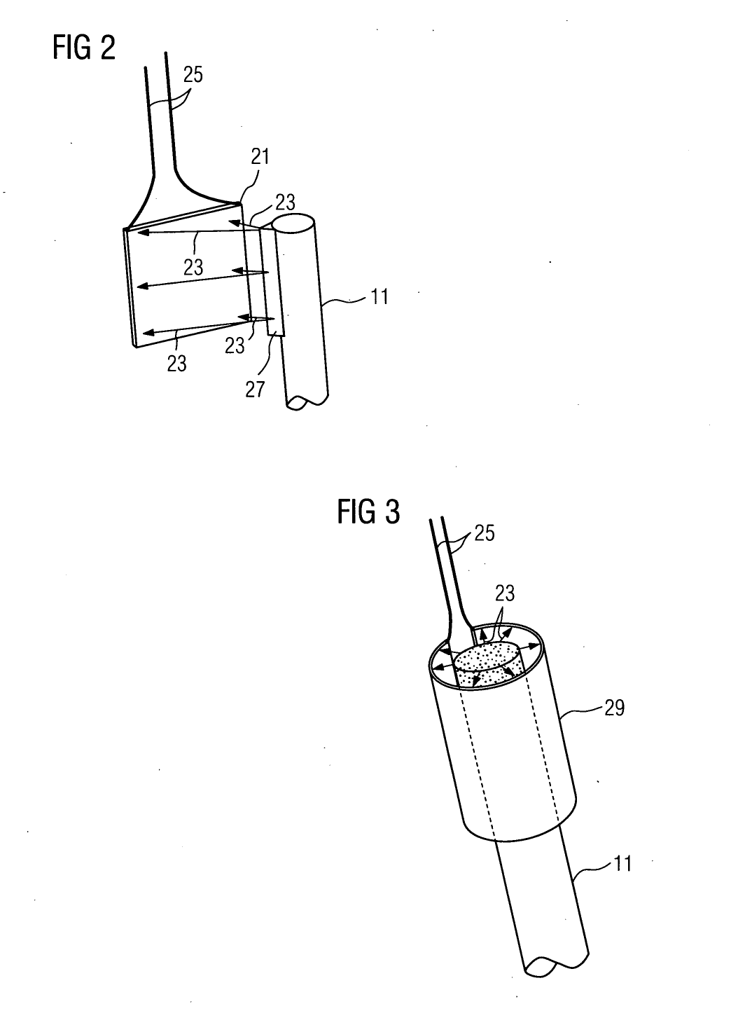

FIG. 2 shows a first embodiment of an electrode 5 with a plane photovoltaic element 21. The light 23 leaving from the light-conducting cable 11 is deflected such that it falls onto the plane photovoltaic element 21. This can be possible for instance by means of a special guiding of the individual fiber optic cables in the electrode region of the light-conducting cable, if the light-conducting cable comprises several fiber optic cables. Small mirror constructions or prism constructions 27 can however also be used on the end of the light-conducting cable so as to direct the light 23 leaving from the light-conducting cable 11 onto the photovoltaic element 21. A further simple and cost-effective solution is to cauterize or break the fiber ends of the fiber optic cable so as to achieve an applicator which scatters in a diffused fashion. This form of light rerouting from a light conductor is already used in laser-induced tumor therapy. The electric voltage generated by the light 23 is applied to the tissue via two electrode tips 25 affixed to the plane photovoltaic element 21.

FIG. 3 shows a further embodiment of the electrode 5. The photovoltaic element 29 is bent coaxially around the light-conducting cable 11. Here the light 23 is also rerouted from the end of the light-conducting cable to the photovoltaic element 29, preferably over a cauterized light conductor on the fiber end. The generated voltage is emitted to the tissue via two electrode tissues 25.

In both embodiments the photovoltaic element 21, 29 and the end of the light-conducting cable 11 are disposed in a protective housing (not shown here), into which the light-conducting cable enters and out of which the electrode tips 25 leave.

1-8. (canceled)

9. A device for electrically stimulating parts of the nervous system comprising:
a generator configured to generate energy;
an intracoporeal electrode configured to be implanted in a human body; and
a MRT-compatible coupling which couples the generator and intracorporeal electrode in a manner that enables energy transfer from the generator to the electrode and which retains the generator and electrode in electrical isolation.

10. The device according to claim 9, wherein,
the electrode comprises a photovoltaic element,
the MRT-compatible coupling comprises a light-conducting cable, and
the generator comprises means for controlling the photovoltaic element of the electrode via light in order to generate voltage, wherein the light-conducting cable is configured to guide light from the generator via the light conducting cable to the electrode in order to generate voltage.

11. The device according to claim 10, the photovoltaic element further comprises, a plane surface configured to be radiated by the light leaving the light-conducting cable.

12. The device according to claim 10, the photovoltaic element further comprises, a curved surface configured and positioned with the light-conducting cable, with the surface curving axially around the light-conducting cable, and wherein the surface is configured to be radiated by light leaving from the light-conducting cable.

13. The device according to claim 10, wherein the generator comprises, means for controlling the light intensity in order to adjust the strength of the electrode voltage.

14. The device according to claim 10, wherein the generator comprises, a light-emitting diode for generating the light.

15. The device according to claim 10, wherein the generator comprises, means for generating the light as a continuous light signal.

16. The device according to claim 10, wherein the generator comprises, means for generating the light as an impulsed light signal.

17. The device according to claim 9, wherein the incorporeal electrode is configured for implantation in the human brain to stimulate regions of the brain.

18. The device according to claim 9, wherein the generator is configured to be implanted intracorporeally.

19. A method for electrically stimulating parts of the nervous system through a device comprising a generator, an intracorporeal electrode and a MRT-compatible coupling, the method comprising:
coupling the generator and an intracoporeal electrode via a MRT-compatible coupling in a manner that enables energy transfer from the generator to the electrode and electrically isolates the generator and intracorporeal electrode;
transferring energy from the generator to the electrode for stimulating parts of the nervous system via the MRT-compatible coupling while maintaining the generator and intracorporeal electrode in electrical isolation; and
generating voltage in the electrode with the energy transferred to the electrode for stimulating parts of the nervous system.

20. The method according to claim 19, wherein the coupling step further comprises coupling a generator with means for providing light to the electrode and an intracorporeal electrode comprising a photovoltaic cell via the MRT-compatible coupling in a manner that enables light to transfer from the generator to the electrode;
the transferring step comprising transferring light from the generator to the electrode via the MRT-compatible coupling;
the voltage generating step comprising generating voltage in the electrode via the light transferred from the generator for stimulating parts of the nervous system.

21. The method according to claim 20, wherein the method further comprises:
in the transferring step, transferring a first level of light intensity from the generator to the electrode via the MRT-compatible coupling and in the generating step, generating a first level of voltage in the electrode for stimulating parts of the nervous system;
adjusting the light intensity in the generator to a second level of light intensity different from first level of light intensity;
transferring light at the second level of light intensity to the electrode via the MRT-compatible coupling; and
generating a second level of voltage in the electrode different from the first level of voltage for stimulating parts of the nervous system.

22. The method according to claim 20, wherein the transferring step further comprises, guiding light from the generator via the MRT-compatible coupling comprising a light-conducting cable to the electrode to generate a predetermined level of voltage in the electrode for simulating parts of the nervous system.

23. The method according to claim 21, wherein the transferring step comprises radiating light from the light-conducting cable of the MRT-compatible coupling onto the surface of the photovoltaic element of the electrode.

24. The method according to claim 22, wherein the transferring step comprises, radiating light coaxially from the light-conducting cable onto the surface of the light-conducting cable.

25. The method according to claim 19, wherein the method further comprises after the voltage generating step;
stimulating parts of the nervous system in the brain via voltage produces in the intracorporeal electrode.

26. The method according to claim 25, wherein the stimulating step comprises stimulating parts of the nervous system in the brain.
